(19) United States
(12) Patent Application Publication
(10) Pub. No.: US 2015/0127097 A1
(43) Pub. Date: May 7, 2015

(54) IMPLANTABLE DEVICE FOR IMPROVING OR RECTIFYING A HEART VALVE INSUFFICIENCY

(71) Applicant: UNIVERSITÄT DUISBURG-ESSEN, Essen (DE)

(72) Inventors: Till Neumann, Bochum (DE); Raimund Erbel, Essen (DE)

(21) Appl. No.: 14/397,465

(22) PCT Filed: May 22, 2013
(86) PCT No.: PCT/EP2013/001506

§ 371 Date: Oct. 27, 2014

(30) Foreign Application Priority Data
Jun. 1, 2012 (DE) 10 2012 010 798.6

(51) Int. Cl. A61F 2/24

(52) U.S. Cl. CPC A61F 2/2409
USPC 623/2.17

(57) ABSTRACT

The invention relates to an implantable device for improving or rectifying a heart valve insufficiency, comprising a closure element (1) which can be positioned in the passage area of a heart valve, in particular in the area between an atrium (2) and a ventricle (3) of the heart, and which has an upper, upstream end (1a) and a lower, downstream end (1b), wherein at least one contact strip (4) is arranged at at least one of the ends (1a, 1b) of the closure element (1), which extends away from the closure element (1) and is led back to the closure element (1) in at least one loop, wherein at least a part of the extent of the strip can be placed against the inner wall of the heart.

The invention relates to an implant for improving or rectifying a heart-valve insufficiency, comprising a closure body that is positionable in a passage of a heart valve, in particular in the region between an atrium and a ventricle of the heart and that has an upper, upstream end and a lower, downstream end.

Devices of this type are known in the prior art, for example, from the publication U.S. Pat. No. 7,785,3662. The device described there has a closure body that has a mitral valve in the passage region and is therefore between the left atrium and the left ventricle of the heart. For this purpose, the closure body is fastened in the myocardium of the heart with a downwardly extending anchor element. The anchor element has a length selected such that the closure body is inside the heart valve, i.e. is enclosed thereby. The device described here has the disadvantage that an implantation in the myocardium is essential for fastening the closure body that is accompanied by a risk of infection and tissue damage. The closure body according to this construction is also only anchored on one side and can move freely around the implantation location at the anchor end. There is no fixing in place within the heart valve.

In general, closure bodies of this type are usable to rectify or at least improve heart valve insufficiencies, i.e. those pathological changes or malformations of the heart valves that have the result that the heart valves do not completely close, but rather a gap remains between the valve cusps even in the closed state, through which blood can flow opposite to the actual flow direction. A heart-valve insufficiency can therefore result in an undersupply of oxygen, thus a lower capacity, and in the worst case in death.

A closure body of the type mentioned above is therefore provided to fill up the gap left in the closed state of a heart valve and to close this gap in this way, or at least reduce it in size, so that the heart valve recovers its function in the closed state.

A closure body of the type in question mentioned above is not restricted to the application mentioned in the prior art in the event of an insufficiency of the mitral valve. Use can also be appropriate in other valves of the heart, for example, in the tricuspid valve, the pulmonary valve, or the aortic valve.

It is the object of the invention to provide a device that is implantable in the heart, with a closure body that is implantable in a simple manner and is positionable and fastenable in the heart in a careful manner and that preferably securely maintains the positioning.

This object is achieved according to the invention in that in a device of the type mentioned above, at least one contact strip is arranged on at least one of the ends of the closure body that extends away from the closure body and is led back to the closure body in at least one loop and can be applied to the inner wall of the heart along at least a part of its extension.

The essential core idea of the invention is that fastening, i.e. fixed positioning of the closure body, is no longer achieved by an implantation in the myocardium, although such a fastening can also still be provided in a refinement in addition to the type of fastening according to the invention.

The fastening according to the invention is performed in that the contact strip that forms at least one loop can be applied to the inner heart wall and contacts the heart wall after an implantation in this case along at least a part of its loop circumference. The at least one loop is therefore guided along the inner wall of the heart.

In this case, it can be provided in a refinement that a contact strip branches on the path of its extension into at least two contact strip arms that are guided back to the closure body. Such a branching can take place, for example, at a location of maximum distance of the branching location from the closure body.

A design according to the invention can also provide that, originating from the closure body, multiple contact strip arms extend on a looped or curved path and meet at a shared connection point and are guided together there. Such a design uses a cage of multiple contact strip arms that are fastened at one of their ends on the closure body and are fastened to one another at their other ends.

In a preferred embodiment, a contact strip is implemented from a spring-elastic material, in particular a spring-elastic material made of a shape-memory alloy.

The spring-elastic implementation has the advantage that a contact of the at least one contact strip on the inner heart wall can occur under the force load that is generated by an internal spring force. For example, the contact strip length or loop length or the size of a cage formed from multiple contact strip arms can be selected to be greater than the selected atrium or the selected ventricle, in which the contact strip is to be fastened.

In such a case, a contact strip or cage is compressed by the heart wall that generates a spring force acting opposite to the compression in the contact strip or cage. A good friction lock and form fit are achieved in this manner. This embodiment also has the advantage that due to the spring-elasticity, the contraction of the heart is not impaired by the device according to the invention.

The formation of the at least one contact strip from a spring-elastic material, in particular a shape-memory alloy, for example, nitinol, has the further advantage that the at least one contact strip can be collapsed, for example, for the purpose of implantation through a catheter, and automatically unfolds after the implantation, i.e. after it is expelled from the catheter, in particular under the effect of body heat. Such a collapsibility can be achieved not only by shape-memory alloys, but rather also by suitable biocompatible spring steel alloys.

The at least one contact strip according to the invention can also be implemented in one possible embodiment that provides spring elasticity, by a wire wound in a helix that has a hollow passage extending longitudinally of the contact strip due to its winding. Such an internally open contact strip can also be formed by an internally hollow wire braid. This wound wire or the wire braid can additionally be embedded in a biocompatible material, for example, in silicone, in particular a material that also encloses the closure body or from which the closure body is at least partially formed.

The wire can itself be made from a shape-memory alloy, for example, nitinol. Alternatively or additionally, a further wire can also be guided inside the wire wound in a helix/wire braid that thus forms a passage, for example, made of a shape-memory alloy such as nitinol.

At least one contact strip, or an above-described cage construction, is on at least one of the ends of the closure body of a device according to the invention, but preferably on both ends, i.e. a lower end and an upper end of the closure body.

In a device for rectifying an insufficiency of the mitral valve or also the tricuspid valve, one contact strip or multiple contact strip arms can form at least one loop that is in the atrium of the heart and one contact strip or multiple contact strip arms can form at least one loop that is arranged in a ventricle. The closure body is located between the two loops or cages and is thus positioned and fastened in the passage of the heart valve. In particular, great axial and also great lateral positioning precision perpendicular to the blood flow is also achieved in this way.

To improve the spring properties of a loop that is formed, it can be provided in a refinement applicable to all embodiments that at least one bend or buckle whose tip points toward the closure body is arranged in a loop of the contact strip, in particular in the loop that is associated with the ventricle.

In all embodiments, in a refinement a contact strip can be fastened in or on the lateral regions of the closure body extending between the ends. The contact strip or a contact strip arm is therefore guided in this embodiment on at least a part of its extension along the extension of the closure body and fastened thereon or therein. Instead of a fastening on or in the lateral regions, fastening can also be performed in the closure body, for example, in the middle or a desired location in the internal volume of the closure body.

In the above-mentioned embodiments, a preferred refinement can also be achieved in which a loop above the upper end and a loop below the lower end of the closure body are formed by a single contact strip that is closed per se in particular where the two loops merge into one another through the lateral regions of the closure body or, for example, in the middle through the closure body.

In the case of a cage formed by multiple contact strip arms, arms corresponding to one another can merge into one another through the closure body at the lower and upper ends of the closure body.

In the case of a separate embodiment, upper and lower contact strips can also each be fastened on the frontal upper and lower ends lying in the flow direction.

In the case of an application for treatment of a mitral valve insufficiency, a closure body has an essentially sickle-shaped cross section perpendicular to the flow direction of the blood. In this embodiment, the contact strip, in particular the single contact strip, can lie in the sickle tips of this cross section.

In a similar manner, in an application for treatment of a tricuspid valve insufficiency in which the closure body has an essentially three-armed star-shaped cross section perpendicular to the flow direction, the contact strip arms can lie in the arm tips. A cage shape of the contact strip arms will therefore preferably be used in the case of such an application.

In all applications, an embodiment can also be provided in which at least one of the loops, in particular the loop(s), associated with the atrium, of one of the ends of the closure body is/are formed as convex to the heart wall, and at least one of the loops, in particular that which is/are associated with the ventricle, forms a tip that is distal to the closure body and is oriented toward the heart wall, and in particular can be anchored in the cardiac muscle.

Thus, according to the invention, the upper end of the closure body is fastened by contact of the loop associated with the atrium on the inner heart wall, but the lower end is fixed in the myocardium, essentially as already known in the prior art. Fastening in the heart is thus achieved at least at both ends of the closure body. In addition, movement of the closure body with the blood stream out of the plane of the valve into the ventricle is prevented by the loops in the atrium.

To further improve the fastening on the heart inner wall, in all possible embodiments barbs or nubs can be arranged on the outer sides of the at least one contact strip. A contact strip can claw into the heart wall using these barbs or prevent a position change via the nubs.

It can also be provided in a refinement that is combinable with all embodiments, that a contact strip, in regions of its extension in which it can be applied to the inner heart wall, has a flattened and widened cross section relative to other regions (in particular at the location of the closure body). A surface area enlargement is thus achieved in the contact regions and in this way irritation of the heart wall is reduced.

In a preferred refinement that is also combinable with all embodiments, not only the at least one contact strip or contact strip arm is formed so it can be unfolded from a collapsed or folded state, but rather also the closure body can be unfolded from a folded or collapsed state by internal application of force.

The entire device according to the invention can thus be introduced as one component through a catheter or via a port into the heart, the device being provided in a collapsed or folded state inside the catheter or port and being unfolded after release from the catheter or port. The operation of the unfolding can be achieved in this case, for example, via the elastic structure of the contact strips.

An internal application of force can be produced in that, for example, a fluid can be pumped into the closure body, a swelling medium is provided in the closure body or the closure body consists of such a medium or comprises such a medium, or also by means of spring force of at least one contact strip that is guided through the closure body, in particular through its sides.

In a refinement, the closure body can have a cross-sectional constriction between the upper and lower ends. In this manner, the closure body can be adapted in a manner favorable for flow. In this case, but also independently of the above-mentioned embodiment, the closure body, in particular at least in the plane of the heart valve, is adapted to the specific shape that was previously determined by measurement technology, of a gap between the valve cusps of a closed heart valve of a patient.

For this purpose, for example, the gap in the closed heart valve can be surveyed in a radiological, sonographic, or videographic manner, to then manufacture the closure body specifically for the patient on the basis of the determined measured values.

Independent of the above-described embodiments, the closure body can be formed at least on its surface from a swelling biocompatible material, in particular a hydrogel. In this way, particularly good leak-tightness between the valve cusps and the closure body is achieved in the closed state of the heart valve.

A biocompatible silicone, for example, can also be selected for the closure body as a further material that ensures good leak resistance. The closure body can have a coating made of such a silicone or also can be entirely manufactured therefrom.

In one embodiment a shaping wire braid is provided in the closure body that is embedded/extrusion coated using silicone or another biocompatible material. In particular, the mentioned biocompatible material/silicone also encloses the at least one contact strip, a total coating of the device according to the invention with the biocompatible material thus resulting.

In a refinement that is combinable with all other embodiments, at least the closure body, and optionally also the at least one contact strip, can have a hydrophilic surface coating or texture. The surface of the closure body and/or of the at least one contact strip can also be formed, for example, by coating, such that endothelialization is promoted.

In particular by way of the above-mentioned or other suitable coatings or textures, in the event of repeated engagement of the valve with the closure body, no damage to the valve occurs.

In a further possible refinement that is combinable with all embodiments, a valve element that is movable by the blood stream in particular, is provided on the closure body, in particular on at least one side, preferably on two opposing sides of the closure body. Such a valve element can preferably be formed such that it reduces the cross section of the flow body in the natural, desired flow direction of the blood (for example, from the atrium into the ventricle), for example, in that the valve element is applied to the closure body due to the acting blood stream or a force assistance, and it enlarges the cross section of the closure body in a flow of the blood opposite to the above-mentioned flow direction, i.e. in the event of a flow reversal, for example, in that the valve element is lifted off of the closure body by the flowing blood or is unfolded in this way. Such a valve element can extend, for example, over the entire width of a closure body, in the case of a sickle-shaped closure body, for example, between the sickle tips.

In this embodiment, a natural heart valve can cooperate with at least one movable valve element and cause secure closure of the gap in the heart valve.

Embodiments of the invention will be described hereafter. In the figures:

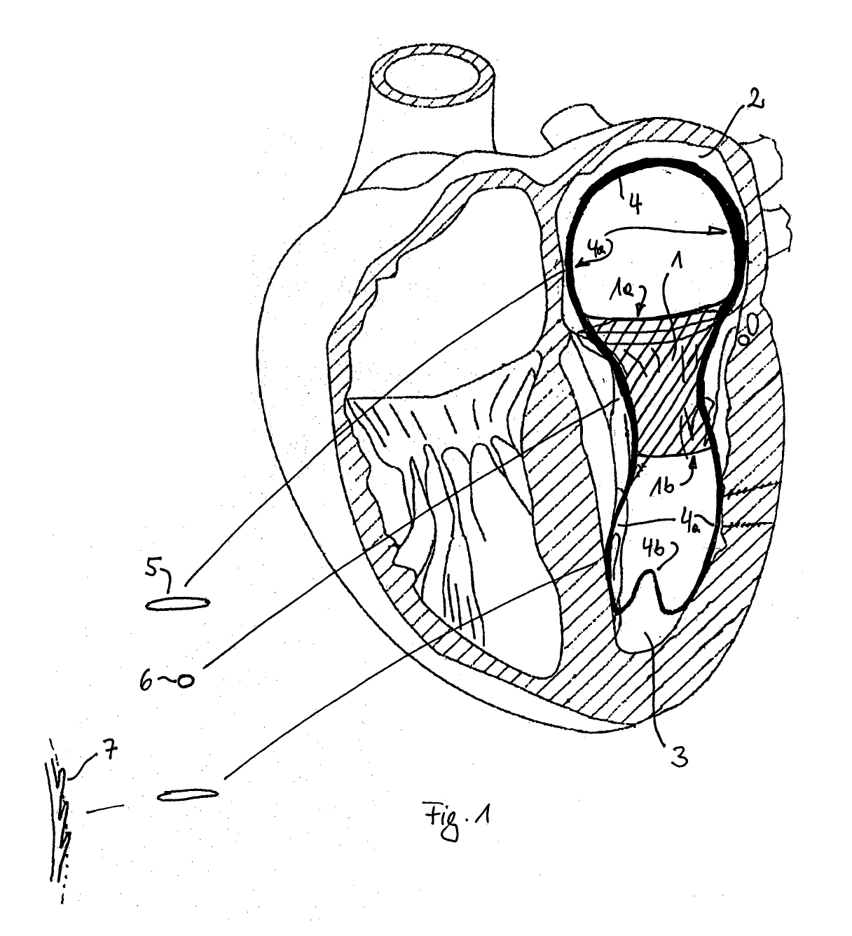

FIG. 1 shows an embodiment with a convex looped contact strip on the upper and lower ends of a closure body for the mitral valve,

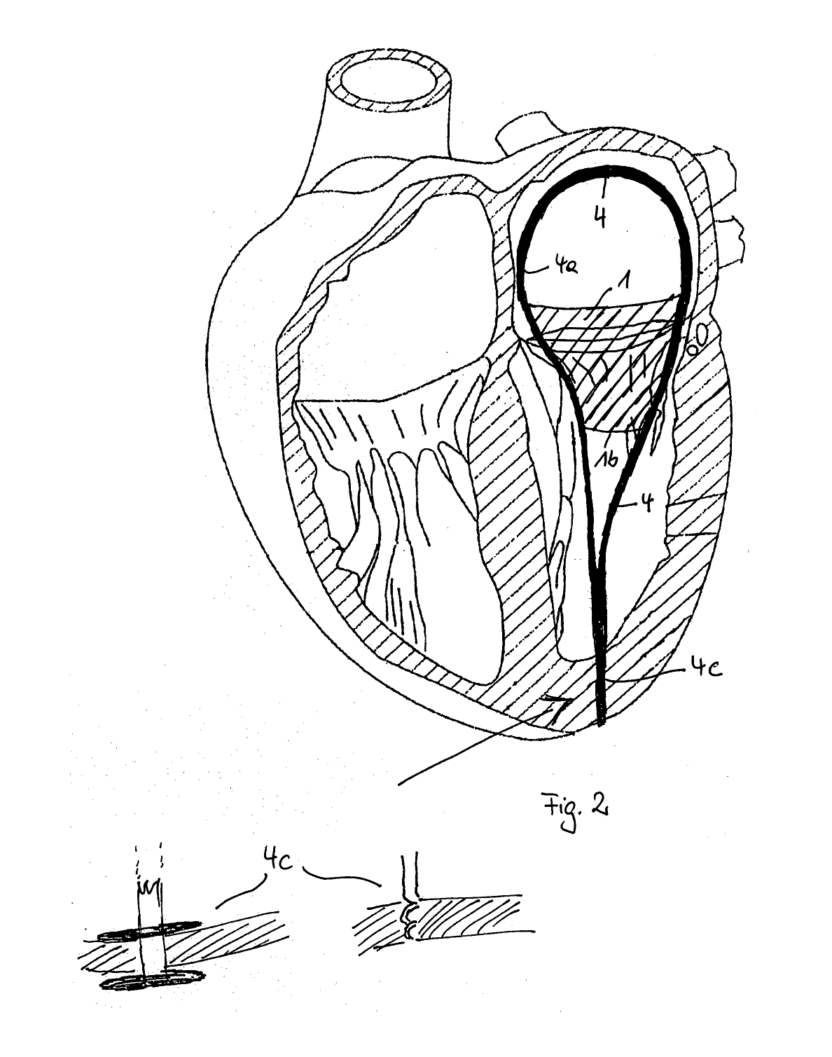

FIG. 2 shows an embodiment with an upper convex looped contact strip and a lower loop that tapers to a point, of the contact strip,

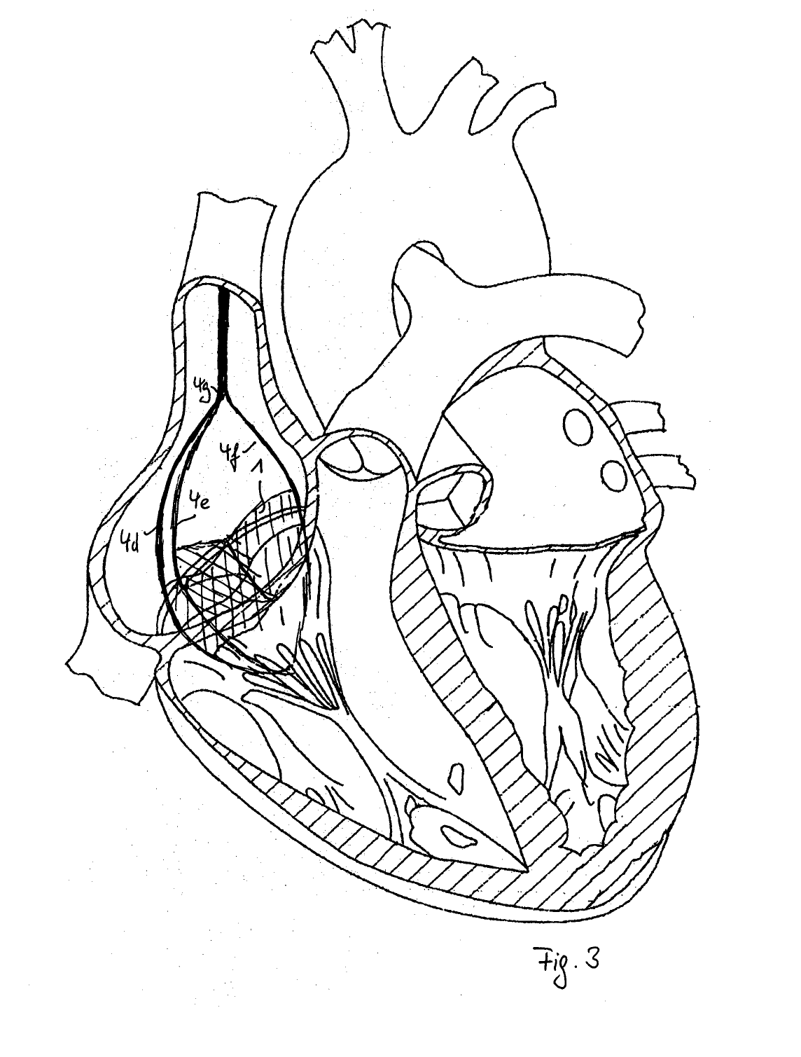

FIG. 3 shows an embodiment for the tricuspid valve with three contact strip arms in each case on the upper and lower ends,

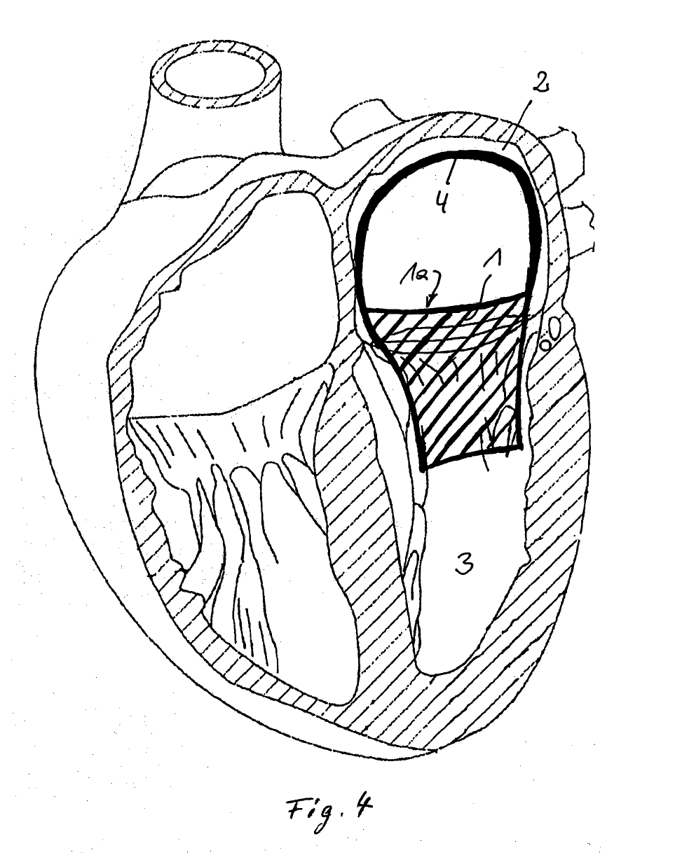

FIG. 4 shows an embodiment with a convex looped contact strip on the upper end of the closure body for the mitral valve,

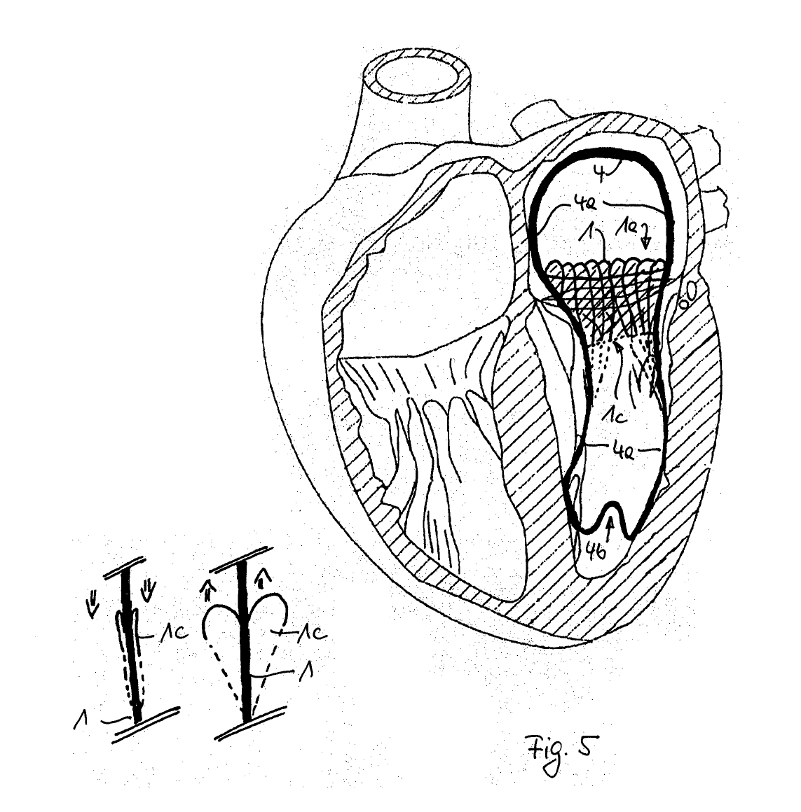

FIG. 5 shows an embodiment for the mitral valve with the use of a valve element on the closure body,

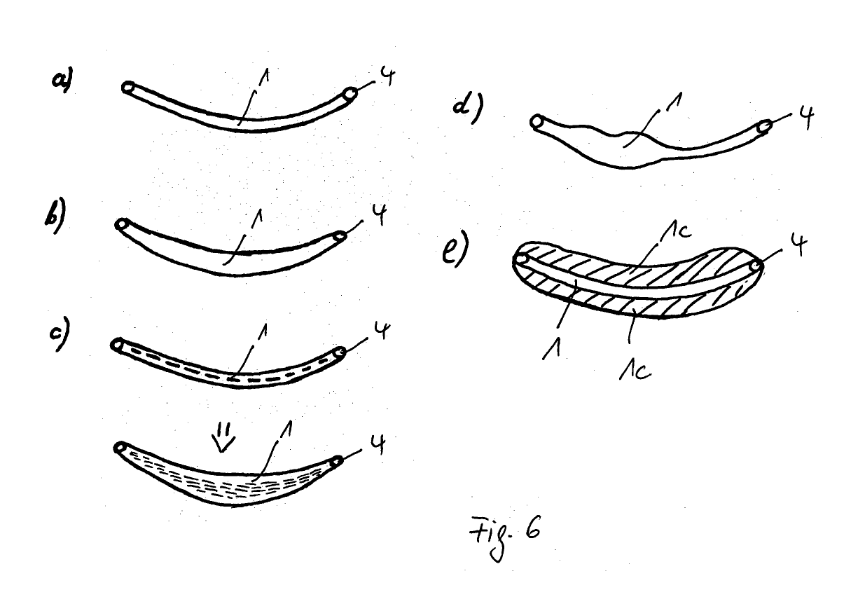

FIG. 6 shows sickle-shaped cross sections of the closure body for a mitral valve insufficiency.

FIG. 1 shows a first embodiment of the device according to the invention for the treatment of mitral-valve insufficiency. The closure body 1 is positioned between an atrium 2 and a ventricle 3 inside the mitral valve, i.e. enclosed by the mitral valve. The cross-sectional shape of the closure body 1 is preferably adapted to the cross-sectional shape of the remaining gap of the closed mitral valve.

At the upper end 1a of the closure body 1 there is a looped contact strip 4 that is shaped convexly to the heart wall and that extends away from the closure body 1 and leads back in a convex curved loop to the closure body and is fastened in the lateral regions thereof on the closure body 1.

The contact strip 4 contacts the heart wall from the inside in lateral regions 4a. In these regions 4a, the contact strip can have a flattened cross section 5, while in contrast in the remaining regions, in particular the fastening regions on the closure body, the cross section 6 of the contact strip 4 can be round.

At the lower end 1b of the closure body, a contact strip 4 is also provided that completes a looped curved course, fundamentally comparable to the upper loop. The lower loop has a bend here, however, whose tip points toward the closure body. This bend can assist the lateral spring-elasticity between the regions 4a of the lower contact strip 4.

In this embodiment, lower and upper contact strips 4 are identical. The loops at the upper and lower ends 1a or 1b of the closure body 1 merge into one another, in that the contact strip is guided through the lateral regions of the closure body or is guided past them and is fastened on the closure body.

FIG. 1 furthermore shows that in this embodiment the outer side of the contact strip 4 has barbs or nubs 7, in particular at least in the flattened contact regions 4a.

In the embodiment shown in FIG. 1, the fastening in the heart is only performed by a contact of the contact strip 4 on the inner heart wall.

FIG. 2 differs in that the contact strip 4 is guided at the lower end 1b of the closure body 1 in a loop that tapers to a point at the distal end. The loop end 4c that is remote from the closure body 1 can have a fastening region to form an anchor in the myocardium, for example, by a screw thread or by anchor plates that enclose the myocardium.

In FIGS. 1 and 2, the closure body 1 is adapted to the gap of the mitral valve and therefore fundamentally has a sickle shape in cross section perpendicular to the flow direction of the blood, as shown in FIG. 6. The contact strip 4 is preferably guided through the respective sickle tip here.

FIG. 3 shows an embodiment for the treatment of an insufficiency of the tricuspid valve. A contact strip with three contact arms is arranged here on the upper and lower ends of the closure body 1 that stellate with three arms 4d, e, f that are guided together at a joint 4g. The arms 4d, e, f therefore form a quasi-cage that spans the upper and lower ends of the closure body.

In the embodiment shown in FIG. 4, the treatment of an insufficiency of the mitral valve is achieved by a closure body 1 that is provided with a convex looped contact strip 4 only at the upper end of a closure body 1 for the mitral valve.

In the embodiment shown in FIG. 5, the treatment of mitral-valve insufficiency is achieved according to the embodiment shown in FIG. 1. Instead of only a closure body 1, reverse flow is additionally achieved by a valve element 1c that is arranged movably on the closure body 1. The valve element 1c that is applied to the closure body 1, rises up or unfolds with flow reversal and in this way prevents reverse flow of the blood from the ventricle 3 into the atrium 2.

In all of the embodiments shown here, the device according to the invention can be unfolded, for example, to be installed from a catheter or port. As shown in FIGS. 6a and b, the closure body can be tubular with an internal volume filled to unfold the closure body.

According to FIG. 6c, the closure body 1 can also be filled with a swellable material 8, so that it automatically swells up due to blood contact and assumes its required shape. An individual shape of the closure body 1 that is adapted to the valve morphology, according to FIG. 6d is also possible. The shape of the valve gap can be determined by measurement technology, for example, and the closure body 1 can be manufactured on the basis of the determined data.

Expansion of the closure body with at least one valve element 1c on each side according to FIG. 6e, is also possible, each expanding the cross section of the closure body 1 in the event of flow reversal and preventing the reverse flow of the blood.

1. An implant for improving or rectifying a heart-valve insufficiency comprising:
a closure body that is positionable in a passage of a heart valve between an atrium and a ventricle of the heart and that has an upper, upstream end and a lower, downstream end;
at least one contact strip on at least one of the ends of the closure body, extending away from the closure body, guided back in at least one loop to the closure body, and engaged against the inner heart wall along at least a part of its extension.

2. The implant according to claim 1, wherein the contact strip is formed from a spring-elastic shape-memory alloy.

3. The implant according to claim 1, wherein the contact strip is formed by a wire wound in a helix that forms a passage, or by a wire braid that forms a passage extending longitudinally of the contact strip with a further wire of a shape-memory alloy extending inside the longitudinal passage.

4. The implant according to one of the preceding claims, claim 1, wherein the contact strip is formed with at least one bend or buckle whose tip points toward the closure body in the loop of the contact strip, in particular in the loop that in the ventricle.

5. The implant according to claim 1, wherein the closure body is of sickle-shaped cross section having tips and the contact strip is fastened in or on lateral regions of the closure body extending between the ends and extending perpendicular to the flow direction in the sickle tips.

6. The implant according to claim 1, wherein the contact strip is closed and formed with a loop over the upper end and a loop under the lower end of the closure body are formed by a single contact strip, the two loops merging into one another through the lateral regions or through an interior of the closure element.

7. The implant according to claim 6, wherein one of the loops is in the atrium and is formed as convex to the heart wall and the other of the loops is in the ventricle and forms a tip that is distal to the closure body, is oriented toward the heart wall, and that can is anchored in the heart muscle.

8. The implant according to claim 1, wherein the closure body can be unfolded from a folded or collapsed state by an internal application of force by a fluid that can be pumped in, a swelling medium, or by at least one contact strip that is guided through the closure body.

9. The implant according claim 1, wherein at least one contact strip branches along its extension path into at least two contact strip sections at a location of maximum spacing from the branching location from the closure body.

10. The implant according to claim 1, wherein barbs or nubs are arranged on the outer sides of the at least one contact strip.

11. The implant according to claim 1, wherein the contact strip has, in regions of its extension in which it can be applied to the inner heart wall, a cross section that is flattened and widened relative to other regions.

12. The implant according to claim 1, wherein the closure body has a cross-sectional constriction between the upper and lower ends.

13. The implant according to claim 1, wherein the closure body is adapted at least in the plane of the heart valve, to a specific shape that was previously determined by measurement technology of a gap between the cusps of a closed heart valve of a patient.

14. The implant according to claim 1, wherein the closure body is formed at least on its surface from a swelling biocompatible material or the closure body has a hydrophilic surface or a coating/texture of the surface that promotes endothelialization.

15. The implant according to claim 1, wherein the implant is so constructed and designed that can be introduced into a heart in collapsed form through a catheter or a port.

16. The implant according to claim 1, further comprising:
a movable valve element on each side of the closure body.
